(19) United States
(12) Patent Application Publication
(10) Pub. No.: US 2014/0128727 A1
(43) Pub. Date: May 8, 2014

(54) SURGICAL LOCATION MONITORING SYSTEM AND METHOD USING NATURAL MARKERS

(71) Applicant: NAVIDENT TECHNOLOGIES, INC., North Vancouver (CA)

(72) Inventors: Ehud Daon, North Vancouver (CA); Martin Gregory Beckett, Bowen Island (CA)

(73) Assignee: NAVIDENT TECHNOLOGIES, INC., North Vancouver (CA)

(21) Appl. No.: 13/744,967

(22) Filed: Jan. 18, 2013

Related U.S. Application Data
(60) Provisional application No. 61/723,993, filed on Nov. 8, 2012.

(51) Int. Cl. A61B 5/06; A61B 6/03; A61B 8/08; A61B 6/00; A61B 5/00; A61B 8/00; A61B 10/02; A61B 1/24; A61C 3/02; A61B 6/14; A61B 8/13; A61B 1/00; A61C 5/00; A61B 1/04; A61B 1/313; A61B 5/055

(52) U.S. Cl. CPC A61B 5/064; A61B 5/062; A61B 5/055; A61B 6/032; A61B 8/0841; A61B 6/4085; A61B 5/0013; A61B 6/563; A61B 8/565; A61B 10/0233; A61B 1/24; A61C 3/02; A61B 6/145; A61B 5/7225; A61B 6/5217; A61B 8/5223; A61B 8/13; A61B 5/7278; A61B 8/483; A61B 5/742; A61B 5/7475; A61B 6/466; A61B 6/467; A61B 8/466; A61B 8/467; A61B 1/00022; A61B 1/00039; A61B 1/00045; A61B 1/00193; A61B 1/00016; A61C 5/007; A61B 5/0077; A61B 1/04; A61B 1/313
USPC 600/424

(57) ABSTRACT

The present invention involves a surgical hardware and software monitoring system and method allows for surgical planning while the patient is available for surgery, for example while the patient is being prepared for surgery so that the system may model the surgical site. In one embodiment, the model may be used to track contemplated surgical procedures and warn the physician regarding possible boundary violations that would indicate an inappropriate location in a surgical procedure. In another embodiment, the monitoring system may track the movement of instruments during the procedure and in reference to the model to enhance observation of the procedure.

### CROSS REFERENCE TO RELATED APPLICATIONS

The present application claims priority under 35 U.S.C. §119(e) of U.S. Provisional Patent Application Ser. No. 61/723,993 filed on Nov. 8, 2012 titled the same as the present application, the disclosures of which is expressly incorporated by reference herein.

### BACKGROUND OF THE INVENTION

1. Field of the Invention

The invention relates to location monitoring hardware and software systems. More specifically, the field of the invention is that of surgical equipment and software for monitoring surgical conditions.

2. Description of the Related Art

Visual and other sensory systems are known, with such systems being capable of both observing and monitoring surgical procedures. With such observation and monitoring systems, computer aided surgeries are now possible, and in fact are being routinely performed. In such procedures, the computer software interacts with both clinical images of the patient and observed surgical images from the current surgical procedure to provide guidance to the physician in conducting the surgery. For example, in one known system a carrier assembly bears at least one fiducial marker onto an attachment element in a precisely repeatable position with respect to a patient's jaw bone, employing the carrier assembly for providing registration between the fiducial marker and the patient's jaw bone and implanting the tooth implant by employing a tracking system which uses the registration to guide a drilling assembly. With this relatively new computer implemented technology, further improvements may further advance the effectiveness of surgical procedures.

### SUMMARY OF THE INVENTION

The present invention is a surgical monitoring system comprising a tracker having sensory equipment for observing proximate a surgical site and a computing device, the computing device characterized by being in communication with the tracker and by having software capable of recognizing a natural fiducial reference in image information obtained by the tracker from the surgical site based on previous scan data of the surgical site; and calculating a model of the surgical site based the image information and on a previous scan of the surgical site.

The surgical monitoring system may further comprise a tracking marker affixed rigidly proximate the surgical site and the software may further be capable of determining a three-dimensional location and orientation of the tracking marker in the image information; and capable of determining from the three-dimensional location and orientation of the tracking marker a three-dimensional location and orientation of the natural fiducial reference in the image information.

The surgical monitoring system may comprise a tracker for obtaining image information of the surgical site and a controller, characterized in that the controller is configurable for obtaining image information of the surgical site from the tracker; identifying the natural fiducial reference in the image information and in previously obtained scan data of the surgical site; determining the three-dimensional location and orientation of the natural fiducial reference in the image information and in the scan data; comparing the three-dimensional location and orientation of the natural fiducial reference in the image information with the three-dimensional location and orientation of the natural fiducial reference in the scan data to spatially relate in three-dimensions the image information to the scan data; and deriving from the comparing a three-dimensional spatial transformation matrix that relates a three-dimensional coordinate in the image information to a corresponding three-dimensional coordinate in the scan data.

The surgical monitoring system may comprise at least one object bearing a tracking marker, wherein the tracker is configured for identifying within the image information the tracking marker and determining the three-dimensional location and orientation of the at least one object from information about the tracking marker. The tracker may be attached to one of the at least one objects. The controller may be configurable for deriving the three-dimensional spatial transformation matrix in real time.

The surgical monitoring system as described above may comprise at least one item or instrument not bearing any markers, with the tracker configured for identifying within the image information the at least one item or instrument based on pre-programmed data describing the three-dimensional shape of the at least one item or instrument; and determining the three-dimensional location and orientation of the at least one item or instrument based on the pre-programmed data.

In a further aspect there is presented a method for relating in real time the three-dimensional location and orientation of a surgical site to the location and orientation of the surgical site in a scan of the surgical site, the method characterized performing a scan of the surgical site to obtain scan data; determining the three-dimensional location and orientation of a natural fiducial reference from the scan data; obtaining real time image information of the surgical site; determining in real time the three-dimensional location and orientation of the natural fiducial reference from the image information; deriving a spatial transformation matrix for expressing in real time the three-dimensional location and orientation of the natural fiducial reference as determined from the image information in terms of the three-dimensional location and orientation of the natural fiducial reference as determined from the scan data.

The method may comprise obtaining image information of the surgical site from the tracker; identifying a natural fiducial reference in the image information and in previously obtained scan data of the surgical site; determining the three-dimensional location and orientation of the natural fiducial reference in the image information and in the scan data, comparing the three-dimensional location and orientation of the natural fiducial reference in the image information with the three-dimensional location and orientation of the natural fiducial reference in the scan data to spatially relate in three-dimensions the image information to the scan data, and deriving from the comparing a three-dimensional spatial transformation matrix that relates a three-dimensional coordinate in the image information to a corresponding three-dimensional coordinate in the scan data.

The method may comprise modeling the surgical site. The method may further comprise identifying within the image information a tracking marker attached to at least one instrument proximate the surgical site; and determining the three-dimensional location and orientation of the at least one object from information about the tracking marker. The method may yet further comprise issuing an alert when the at least one instrument is determined to be proximate an inappropriate location within the surgical site.

The method may further comprise deriving the three-dimensional spatial transformation matrix in real time by obtaining refreshed image information in real time.

The method may further comprise determining in the image information a three-dimensional location and an orientation of a tracking marker rigidly attached to the surgical site; and determining from the three-dimensional location and orientation of the tracking marker the three-dimensional location and orientation of the natural fiducial reference in the image information.

In another aspect a method is provided for tracking at least one item or instrument proximate a surgical site, comprising the aforementioned method for relating the three-dimensional location and orientation of the surgical site to the location and orientation of the surgical site in a scan of the surgical site; further comprising identifying within the image information the at least one object based on pre-programmed data describing the three-dimensional shape of the at least one object; and determining the three-dimensional location and orientation of the at least one object relative to the three-dimensional location and orientation of the surgical site based on the pre-programmed data. In such an embodiment no tracking marker is required on the item or instrument.

### BRIEF DESCRIPTION OF THE DRAWINGS

The above mentioned and other features and objects of this invention, and the manner of attaining them, will become more apparent and the invention itself will be better understood by reference to the following description of an embodiment of the invention taken in conjunction with the accompanying drawings, wherein:

FIG. 1 is a schematic diagrammatic view of a network system in which embodiments of the present invention may be utilized.

FIG. 2 is a block diagram of a computing system (either a server or client, or both, as appropriate), with optional input devices (e.g., keyboard, mouse, touch screen, etc.) and output devices, hardware, network connections, one or more processors, and memory/storage for data and modules, etc. which may be utilized as controller and display in conjunction with embodiments of the present invention.

FIGS. 3A-J are drawings of hardware components of the surgical monitoring system according to embodiments of the invention.

FIGS. 4A-C is a flow chart diagram illustrating one embodiment of the registering method of the present invention.

FIG. 5 is a drawing of a dental fiducial key with a tracking pole and a dental drill according to one embodiment of the present invention.

FIG. 6 is a drawing of an endoscopic surgical site showing the fiducial key, endoscope, and biopsy needle according to another embodiment of the invention.

FIG. 7 is a drawing of a surgical monitoring system according to a further embodiment of the invention.

FIG. 8 is a flow chart diagram illustrating a further embodiment of the registering method of the present invention employing the surgical monitoring system of FIG. 7.

Corresponding reference characters indicate corresponding parts throughout the several views. Although the drawings represent embodiments of the present invention, the drawings are not necessarily to scale and certain features may be exaggerated in order to better illustrate and explain the present invention. The flow charts and screen shots are also representative in nature, and actual embodiments of the invention may include further features or steps not shown in the drawings. The exemplification set out herein illustrates an embodiment of the invention, in one form, and such exemplifications are not to be construed as limiting the scope of the invention in any manner.

### DESCRIPTION OF EMBODIMENTS OF THE PRESENT INVENTION

The embodiments disclosed below are not intended to be exhaustive or limit the invention to the precise form disclosed in the following detailed description. Rather, the embodiments are chosen and described so that others skilled in the art may utilize their teachings.

The detailed descriptions that follow are presented in part in terms of algorithms and symbolic representations of operations on data bits within a computer memory representing alphanumeric characters or other information. The hardware components are shown with particular shapes and relative orientations and sizes using particular scanning techniques, although in the general case one of ordinary skill recognizes that a variety of particular shapes and orientations and scanning methodologies may be used within the teaching of the present invention. A computer generally includes a processor for executing instructions and memory for storing instructions and data, including interfaces to obtain and process imaging data. When a general-purpose computer has a series of machine encoded instructions stored in its memory, the computer operating on such encoded instructions may become a specific type of machine, namely a computer particularly configured to perform the operations embodied by the series of instructions. Some of the instructions may be adapted to produce signals that control operation of other machines and thus may operate through those control signals to transform materials far removed from the computer itself. These descriptions and representations are the means used by those skilled in the art of data processing arts to most effectively convey the substance of their work to others skilled in the art.

An algorithm is here, and generally, conceived to be a self-consistent sequence of steps leading to a desired result. These steps are those requiring physical manipulations of physical quantities, observing and measuring scanned data representative of matter around the surgical site. Usually, though not necessarily, these quantities take the form of electrical or magnetic pulses or signals capable of being stored, transferred, transformed, combined, compared, and otherwise manipulated. It proves convenient at times, principally for reasons of common usage, to refer to these signals as bits, values, symbols, characters, display data, terms, numbers, or the like as a reference to the physical items or manifestations in which such signals are embodied or expressed to capture the underlying data of an image. It should be borne in mind, however, that all of these and similar terms are to be associated with the appropriate physical quantities and are merely used here as convenient labels applied to these quantities.

Some algorithms may use data structures for both inputting information and producing the desired result. Data structures greatly facilitate data management by data processing systems, and are not accessible except through sophisticated software systems. Data structures are not the information content of a memory, rather they represent specific electronic structural elements that impart or manifest a physical organization on the information stored in memory. More than mere abstraction, the data structures are specific electrical or magnetic structural elements in memory, which simultaneously represent complex data accurately, often data modeling physical characteristics of related items, and provide increased efficiency in computer operation.

Further, the manipulations performed are often referred to in terms, such as comparing or adding, commonly associated with mental operations performed by a human operator. No such capability of a human operator is necessary, or desirable in most cases, in any of the operations described herein that form part of the present invention; the operations are machine operations. Useful machines for performing the operations of the present invention include general-purpose digital computers or other similar devices. In all cases the distinction between the method operations in operating a computer and the method of computation itself should be recognized. The present invention relates to a method and apparatus for operating a computer in processing electrical or other (e.g., mechanical, chemical) physical signals to generate other desired physical manifestations or signals. The computer operates on software modules, which are collections of signals stored on a media that represents a series of machine instructions that enable the computer processor to perform the machine instructions that implement the algorithmic steps. Such machine instructions may be the actual computer code the processor interprets to implement the instructions, or alternatively may be a higher level coding of the instructions that is interpreted to obtain the actual computer code. The software module may also include a hardware component, wherein some aspects of the algorithm are performed by the circuitry itself rather as a result of an instruction.

The present invention also relates to an apparatus for performing these operations. This apparatus may be specifically constructed for the required purposes or it may comprise a general-purpose computer as selectively activated or reconfigured by a computer program stored in the computer. The algorithms presented herein are not inherently related to any particular computer or other apparatus unless explicitly indicated as requiring particular hardware. In some cases, the computer programs may communicate or relate to other programs or equipment through signals configured to particular protocols, which may or may not require specific hardware or programming to interact. In particular, various general-purpose machines may be used with programs written in accordance with the teachings herein, or it may prove more convenient to construct more specialized apparatus to perform the required method steps. The required structure for a variety of these machines will appear from the description below.

The present invention may deal with “object-oriented” software, and particularly with an “object-oriented” operating system. The “object-oriented” software is organized into “objects”, each comprising a block of computer instructions describing various procedures (“methods”) to be performed in response to “messages” sent to the object or “events” which occur with the object. Such operations include, for example, the manipulation of variables, the activation of an object by an external event, and the transmission of one or more messages to other objects. Often, but not necessarily, a physical object has a corresponding software object that may collect and transmit observed data from the physical device to the software system. Such observed data may be accessed from the physical object and/or the software object merely as an item of convenience; therefore where “actual data” is used in the following description, such “actual data” may be from the instrument itself or from the corresponding software object or module.

Messages are sent and received between objects having certain functions and knowledge to carry out processes. Messages are generated in response to user instructions, for example, by a user activating an icon with a “mouse” pointer generating an event. Also, messages may be generated by an object in response to the receipt of a message. When one of the objects receives a message, the object carries out an operation (a message procedure) corresponding to the message and, if necessary, returns a result of the operation. Each object has a region where internal states (instance variables) of the object itself are stored and where the other objects are not allowed to access. One feature of the object-oriented system is inheritance. For example, an object for drawing a “circle” on a display may inherit functions and knowledge from another object for drawing a “shape” on a display.

A programmer “programs” in an object-oriented programming language by writing individual blocks of code each of which creates an object by defining its methods. A collection of such objects adapted to communicate with one another by means of messages comprises an object-oriented program. Object-oriented computer programming facilitates the modeling of interactive systems in that each component of the system may be modeled with an object, the behavior of each component being simulated by the methods of its corresponding object, and the interactions between components being simulated by messages transmitted between objects.

An operator may stimulate a collection of interrelated objects comprising an object-oriented program by sending a message to one of the objects. The receipt of the message may cause the object to respond by carrying out predetermined functions, which may include sending additional messages to one or more other objects. The other objects may in turn carry out additional functions in response to the messages they receive, including sending still more messages. In this manner, sequences of message and response may continue indefinitely or may come to an end when all messages have been responded to and no new messages are being sent. When modeling systems utilizing an object-oriented language, a programmer need only think in terms of how each component of a modeled system responds to a stimulus and not in terms of the sequence of operations to be performed in response to some stimulus. Such sequence of operations naturally flows out of the interactions between the objects in response to the stimulus and need not be preordained by the programmer.

Although object-oriented programming makes simulation of systems of interrelated components more intuitive, the operation of an object-oriented program is often difficult to understand because the sequence of operations carried out by an object-oriented program is usually not immediately apparent from a software listing as in the case for sequentially organized programs. Nor is it easy to determine how an object-oriented program works through observation of the readily apparent manifestations of its operation. Most of the operations carried out by a computer in response to a program are “invisible” to an observer since only a relatively few steps in a program typically produce an observable computer output.

In the following description, several terms that are used frequently have specialized meanings in the present context. The term “object” relates to a set of computer instructions and associated data, which may be activated directly or indirectly by the user. The terms “windowing environment”, “running in windows”, and “object oriented operating system” are used to denote a computer user interface in which information is manipulated and displayed on a video display such as within bounded regions on a raster scanned video display. The terms “network”, “local area network”, “LAN”, “wide area network”, or “WAN” mean two or more computers that are connected in such a manner that messages may be transmitted between the computers. In such computer networks, typically one or more computers operate as a “server”, a computer with large storage devices such as hard disk drives and communication hardware to operate peripheral devices such as printers or modems. Other computers, termed “workstations”, provide a user interface so that users of computer networks may access the network resources, such as shared data files, common peripheral devices, and inter-workstation communication. Users activate computer programs or network resources to create “processes” which include both the general operation of the computer program along with specific operating characteristics determined by input variables and its environment. Similar to a process is an agent (sometimes called an intelligent agent), which is a process that gathers information or performs some other service without user intervention and on some regular schedule. Typically, an agent, using parameters typically provided by the user, searches locations either on the host machine or at some other point on a network, gathers the information relevant to the purpose of the agent, and presents it to the user on a periodic basis.

The term “desktop” means a specific user interface which presents a menu or display of objects with associated settings for the user associated with the desktop. When the desktop accesses a network resource, which typically requires an application program to execute on the remote server, the desktop calls an Application Program Interface, or “API”, to allow the user to provide commands to the network resource and observe any output. The term “Browser” refers to a program which is not necessarily apparent to the user, but which is responsible for transmitting messages between the desktop and the network server and for displaying and interacting with the network user. Browsers are designed to utilize a communications protocol for transmission of text and graphic information over a worldwide network of computers, namely the “World Wide Web” or simply the “Web”. Examples of Browsers compatible with the present invention include the Internet Explorer program sold by Microsoft Corporation (Internet Explorer is a trademark of Microsoft Corporation), the Opera Browser program created by Opera Software ASA, or the Firefox browser program distributed by the Mozilla Foundation (Firefox is a registered trademark of the Mozilla Foundation). Although the following description details such operations in terms of a graphic user interface of a Browser, the present invention may be practiced with text based interfaces, or even with voice or visually activated interfaces, that have many of the functions of a graphic based Browser.

Browsers display information, which is formatted in a Standard Generalized Markup Language (“SGML”) or a HyperText Markup Language (“HTML”), both being scripting languages, which embed non-visual codes in a text document through the use of special ASCII text codes. Files in these formats may be easily transmitted across computer networks, including global information networks like the Internet, and allow the Browsers to display text, images, and play audio and video recordings. The Web utilizes these data file formats to conjunction with its communication protocol to transmit such information between servers and workstations. Browsers may also be programmed to display information provided in an eXtensible Markup Language (“XML”) file, with XML files being capable of use with several Document Type Definitions (“DTD”) and thus more general in nature than SGML or HTML. The XML file may be analogized to an object, as the data and the stylesheet formatting are separately contained (formatting may be thought of as methods of displaying information, thus an XML file has data and an associated method).

The terms “personal digital assistant” or “PDA”, as defined above, means any handheld, mobile device that combines computing, telephone, fax, e-mail and networking features. The terms “wireless wide area network” or “WWAN” mean a wireless network that serves as the medium for the transmission of data between a handheld device and a computer. The term “synchronization” means the exchanging of information between a first device, e.g. a handheld device, and a second device, e.g. a desktop computer, either via wires or wirelessly. Synchronization ensures that the data on both devices are identical (at least at the time of synchronization).

In wireless wide area networks, communication primarily occurs through the transmission of radio signals over analog, digital cellular, or personal communications service (“PCS”) networks. Signals may also be transmitted through microwaves and other electromagnetic waves. At the present time, most wireless data communication takes place across cellular systems using second generation technology such as code-division multiple access (“CDMA”), time division multiple access (“TDMA”), the Global System for Mobile Communications (“GSM”), Third Generation (wideband or “3G”), Fourth Generation (broadband or “4G”), personal digital cellular (“PDC”), or through packet-data technology over analog systems such as cellular digital packet data (CDPD″) used on the Advance Mobile Phone Service (“AMPS”).

The terms “wireless application protocol” or “WAP” mean a universal specification to facilitate the delivery and presentation of web-based data on handheld and mobile devices with small user interfaces. “Mobile Software” refers to the software operating system, which allows for application programs to be implemented on a mobile device such as a mobile telephone or PDA. Examples of Mobile Software are Java and Java ME (Java and JavaME are trademarks of Sun Microsystems, Inc. of Santa Clara, Calif.), BREW (BREW is a registered trademark of Qualcomm Incorporated of San Diego, Calif.), Windows Mobile (Windows is a registered trademark of Microsoft Corporation of Redmond, Wash.), Palm OS (Palm is a registered trademark of Palm, Inc. of Sunnyvale, Calif.), Symbian OS (Symbian is a registered trademark of Symbian Software Limited Corporation of London, United Kingdom), ANDROID OS (ANDROID is a registered trademark of Google, Inc. of Mountain View, Calif.), and iPhone OS (iPhone is a registered trademark of Apple, Inc. of Cupertino, Calif.), and Windows Phone 7. “Mobile Apps” refers to software programs written for execution with Mobile Software.

The terms “scan,” “fiducial reference”, “fiducial location”, “marker,” “tracker” and “image information” have particular meanings in the present disclosure. For purposes of the present disclosure, “scan” or derivatives thereof refer to x-ray, magnetic resonance imaging (MRI), computerized tomography (CT), sonography, cone beam computerized tomography (CBCT), or any system that produces a quantitative spatial representation of a patient. The term “fiducial reference” or simply “fiducial” refers to an object or reference on the image of a scan that is uniquely identifiable as a fixed recognizable point. In the present specification the term “fiducial location” refers to a useful location to which a fiducial reference is attached. A “fiducial location” will typically be proximate a surgical site. The term “marker” or “tracking marker” refers to an object or reference that may be perceived by a sensor proximate to the location of the surgical or dental procedure, where the sensor may be an optical sensor, a radio frequency identifier (RFID), a sonic motion detector, an ultra-violet or infrared sensor. The term “tracker” refers to a device or system of devices able to determine the location of the markers and their orientation and movement continually in ‘real time’ during a procedure. As an example of a possible implementation, if the markers are composed of printed targets then the tracker may include a stereo camera pair. The term “image information” is used in the present specification to describe information obtained by the tracker, whether optical or otherwise, and usable for determining the location of the markers and their orientation and movement continually in ‘real time’ during a procedure.

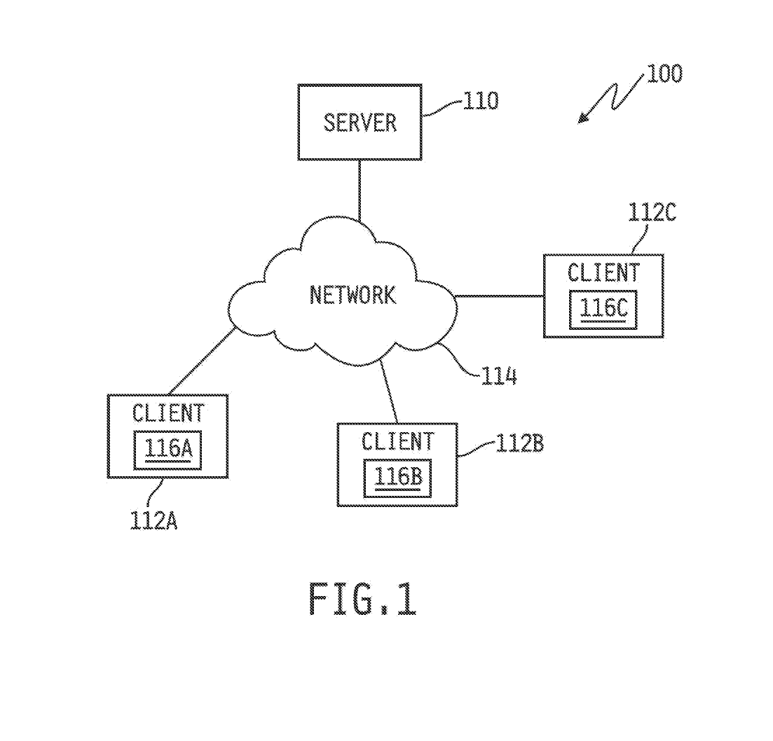

FIG. 1 is a high-level block diagram of a computing environment 100 according to one embodiment. FIG. 1 illustrates server 110 and three clients 112 connected by network 114. Only three clients 112 are shown in FIG. 1 in order to simplify and clarify the description. Embodiments of the computing environment 100 may have thousands or millions of clients 112 connected to network 114, for example the Internet. Users (not shown) may operate software 116 on one of clients 112 to both send and receive messages network 114 via server 110 and its associated communications equipment and software (not shown).

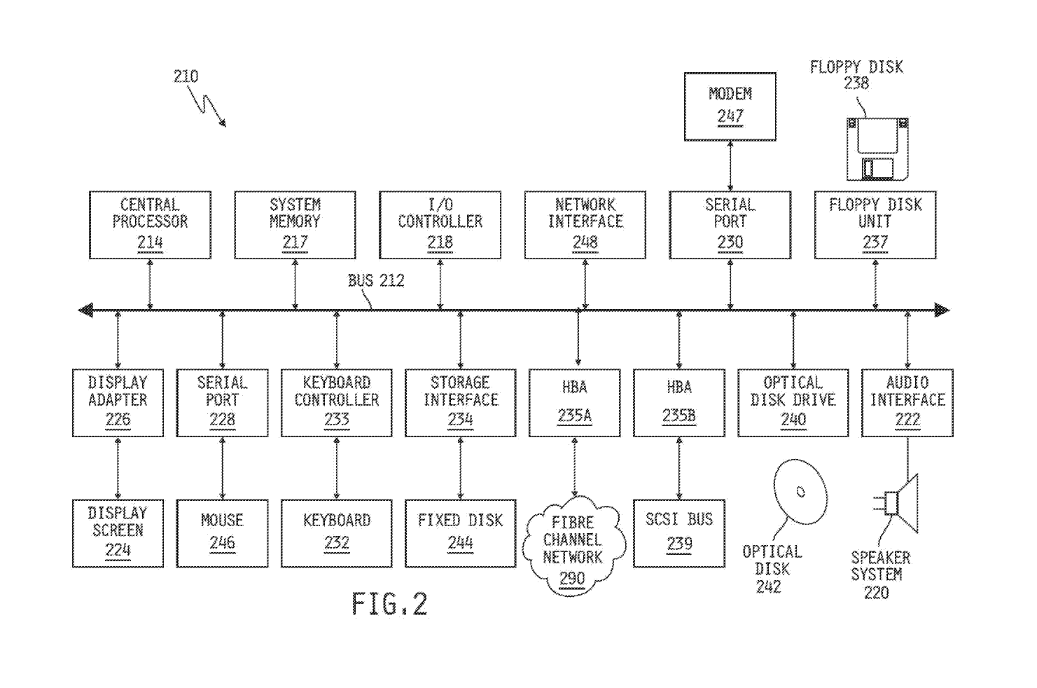

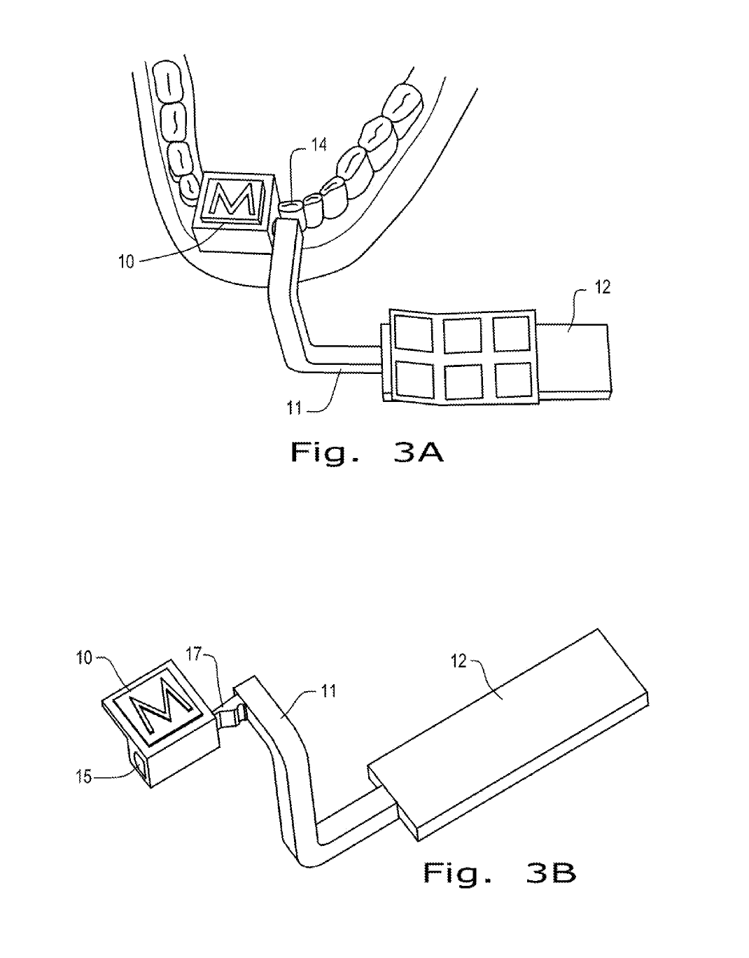

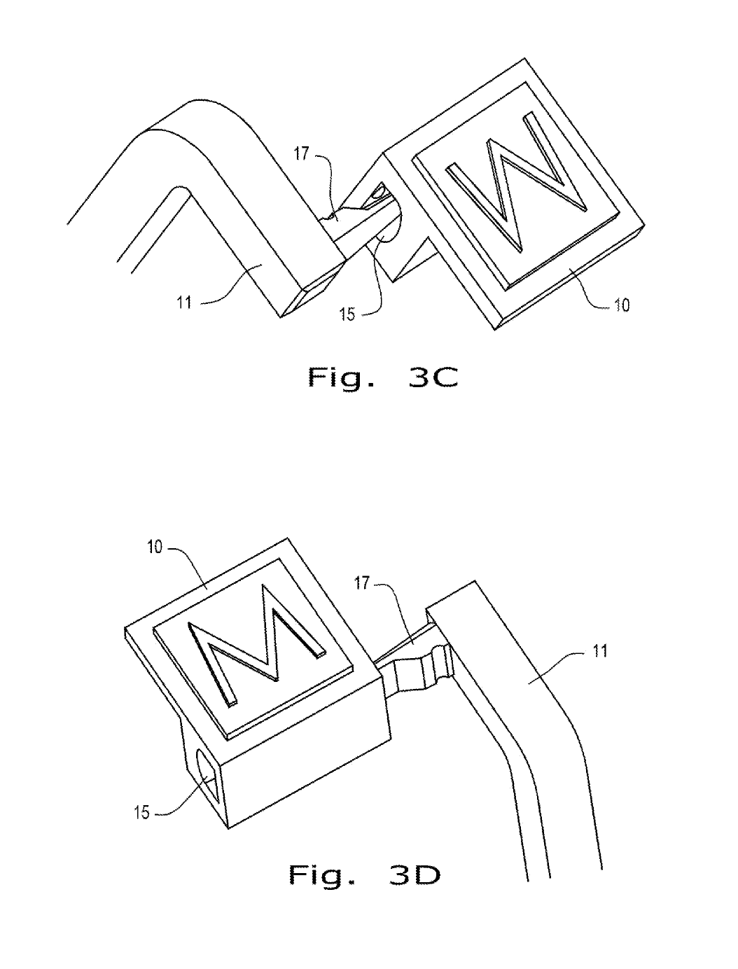

FIG. 2 depicts a block diagram of computer system 210 suitable for implementing server 110 or client 112. Computer system 210 includes bus 212 which interconnects major subsystems of computer system 210, such as central processor 214, system memory 217 (typically RAM, but which may also include ROM, flash RAM, or the like), input/output controller 218, external audio device, such as speaker system 220 via audio output interface 222, external device, such as display screen 224 via display adapter 226, serial ports 228 and 230, keyboard 232 (interfaced with keyboard controller 233), storage interface 234, disk drive 237 operative to receive floppy disk 238, host bus adapter (HBA) interface card 235A operative to connect with Fibre Channel network 290, host bus adapter (HBA) interface card 235B operative to connect to SCSI bus 239, and optical disk drive 240 operative to receive optical disk 242. Also included are mouse 246 (or other point-and-click device, coupled to bus 212 via serial port 228), modem 247 (coupled to bus 212 via serial port 230), and network interface 248 (coupled directly to bus 212).

Bus 212 allows data communication between central processor 214 and system memory 217, which may include read-only memory (ROM) or flash memory (neither shown), and random access memory (RAM) (not shown), as previously noted. RAM is generally the main memory into which operating system and application programs are loaded. ROM or flash memory may contain, among other software code, Basic Input-Output system (BIOS), which controls basic hardware operation such as interaction with peripheral components. Applications resident with computer system 210 are generally stored on and accessed via computer readable media, such as hard disk drives (e.g., fixed disk 244), optical drives (e.g., optical drive 240), floppy disk unit 237, or other storage medium. Additionally, applications may be in the form of electronic signals modulated in accordance with the application and data communication technology when accessed via network modem 247 or interface 248 or other telecommunications equipment (not shown).

Storage interface 234, as with other storage interfaces of computer system 210, may connect to standard computer readable media for storage and/or retrieval of information, such as fixed disk drive 244. Fixed disk drive 244 may be part of computer system 210 or may be separate and accessed through other interface systems. Modem 247 may provide direct connection to remote servers via telephone link or the Internet via an Internet service provider (ISP) (not shown). Network interface 248 may provide direct connection to remote servers via direct network link to the Internet via a POP (point of presence). Network interface 248 may provide such connection using wireless techniques, including digital cellular telephone connection, Cellular Digital Packet Data (CDPD) connection, digital satellite data connection or the like.

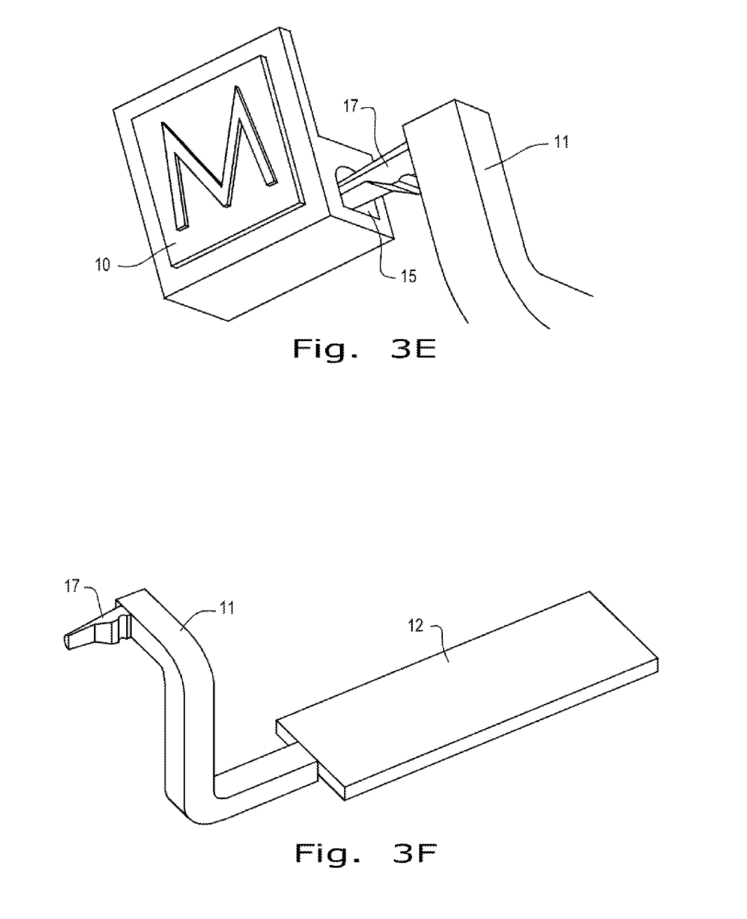

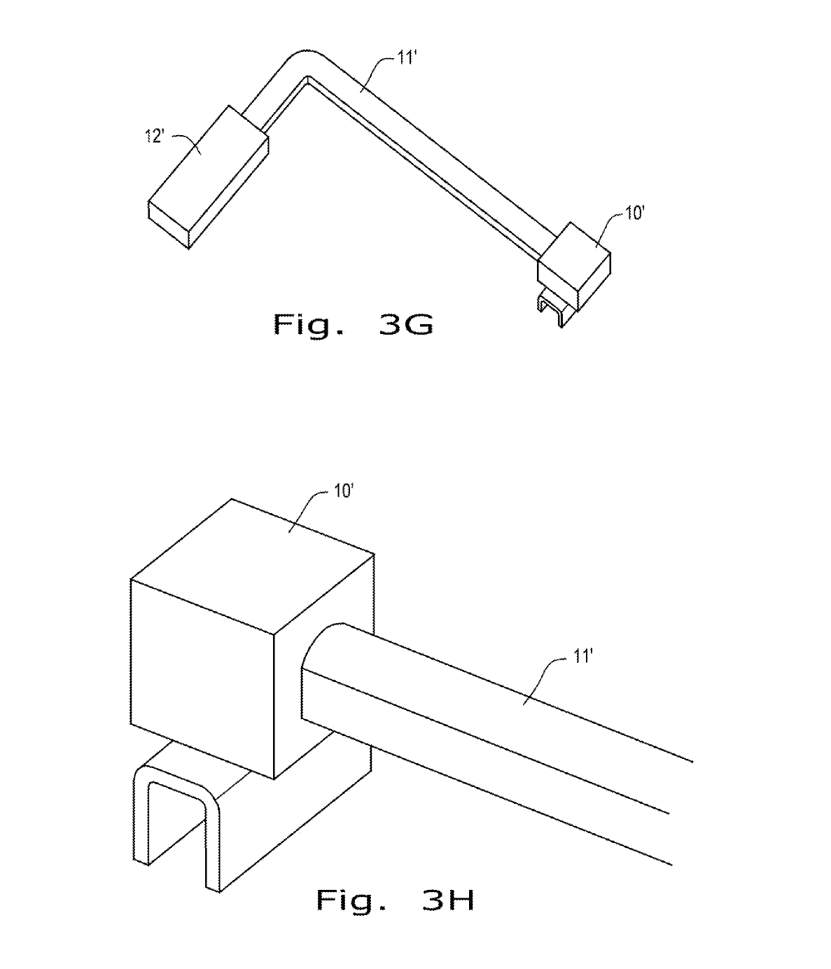

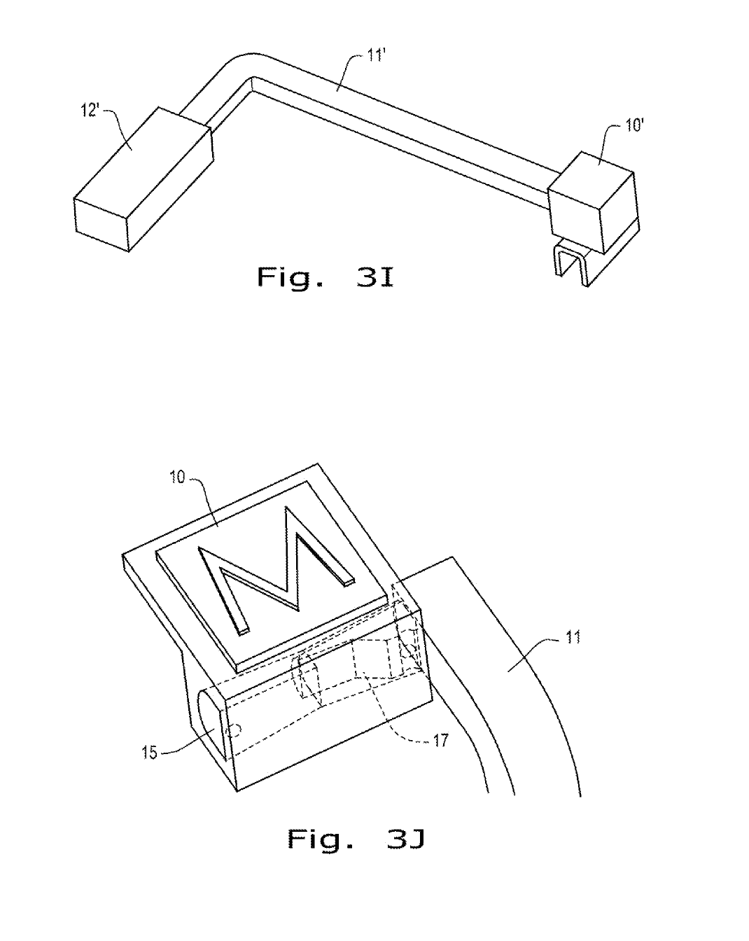

Many other devices or subsystems (not shown) may be connected in a similar manner (e.g., document scanners, digital cameras and so on), including the hardware components of FIGS. 3A-I, which alternatively may be in communication with associated computational resources through local, wide-area, or wireless networks or communications systems. Thus, while the disclosure may generally discuss an embodiment where the hardware components are directly connected to computing resources, one of ordinary skill in this area recognizes that such hardware may be remotely connected with computing resources. Conversely, all of the devices shown in FIG. 2 need not be present to practice the present disclosure. Devices and subsystems may be interconnected in different ways from that shown in FIG. 2. Operation of a computer system such as that shown in FIG. 2 is readily known in the art and is not discussed in detail in this application. Software source and/or object codes to implement the present disclosure may be stored in computer-readable storage media such as one or more of system memory 217, fixed disk 244, optical disk 242, or floppy disk 238. The operating system provided on computer system 210 may be a variety or version of either MS-DOS® (MS-DOS is a registered trademark of Microsoft Corporation of Redmond, Wash.), WINDOWS® (WINDOWS is a registered trademark of Microsoft Corporation of Redmond, Wash.), OS/2® (OS/2 is a registered trademark of International Business Machines Corporation of Armonk, N.Y.), UNIX® (UNIX is a registered trademark of X/Open Company Limited of Reading, United Kingdom), Linux® (Linux is a registered trademark of Linus Torvalds of Portland, Oreg.), or other known or developed operating system.

Moreover, regarding the signals described herein, those skilled in the art recognize that a signal may be directly transmitted from a first block to a second block, or a signal may be modified (e.g., amplified, attenuated, delayed, latched, buffered, inverted, filtered, or otherwise modified) between blocks. Although the signals of the above-described embodiments are characterized as transmitted from one block to the next, other embodiments of the present disclosure may include modified signals in place of such directly transmitted signals as long as the informational and/or functional aspect of the signal is transmitted between blocks. To some extent, a signal input at a second block may be conceptualized as a second signal derived from a first signal output from a first block due to physical limitations of the circuitry involved (e.g., there will inevitably be some attenuation and delay). Therefore, as used herein, a second signal derived from a first signal includes the first signal or any modifications to the first signal, whether due to circuit limitations or due to passage through other circuit elements which do not change the informational and/or final functional aspect of the first signal.

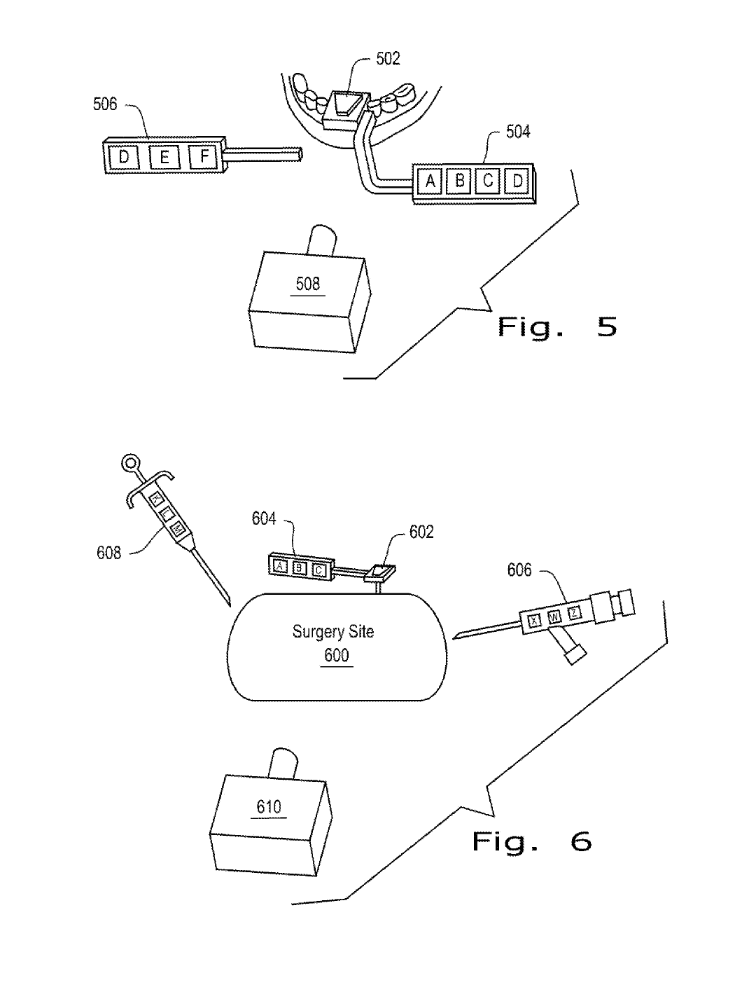

The present invention relates to a surgical hardware and software monitoring system and method which allows for surgical planning while the patient is available for surgery, for example while the patient is being prepared for surgery so that the system may model the surgical site. The system uses a particularly configured piece of hardware, represented as fiducial key 10 in FIG. 3A, to orient tracking marker 12 of the monitoring system with regard to the critical area of the surgery. Fiducial key 10 is attached to a location near the intended surgical area, in the exemplary embodiment of the dental surgical area of FIG. 3A, fiducial key 10 is attached to a dental splint 14. Tracking marker 12 may be connected to fiducial key 10 by tracking pole 11. In embodiments in which the fiducial reference is directly visible to a suitable tracker (see for example FIG. 5 and FIG. 6) that acquires image information about the surgical site, a tracking marker may be attached directly to the fiducial reference. For example a dental surgery, the dental traking marker 14 may be used to securely locate the fiducial 10 near the surgical area. The fiducial key 10 may be used as a point of reference, or a fiducial, for the further image processing of data acquired from tracking marker 12 by the tracker.

In other embodiments additional tracking markers 12 may be attached to items independent of the fiducial key 10 and any of its associated tracking poles 11 or tracking markers 12. This allows the independent items to be tracked by the tracker.

In a further embodiment at least one of the items or instruments near the surgical site may optionally have a tracker attached to function as tracker for the monitoring system of the invention and to thereby sense the orientation and the position of the tracking marker 12 and of any other additional tracking markers relative to the scan data of the surgical area. By way of example, the tracker attached to an instrument may be a miniature digital camera and it may be attached, for example, to a dentist's drill. Any other markers to be tracked by the tracker attached to the item or instrument must be within the field of view of the tracker.

Using the dental surgery example, the patient is scanned to obtain an initial scan of the surgical site. The particular configuration of fiducial key 10 allows computer software stored in memory and executed in a suitable controller, for example processor 214 and memory 217 of computer 210 of FIG. 2, to recognize its relative position within the surgical site from the scan data, so that further observations may be made with reference to both the location and orientation of fiducial key 10. In some embodiments, the fiducial reference includes a marking that is apparent as a recognizable identifying symbol when scanned. In other embodiments, the fiducial reference includes a shape that is distinct in the sense that the body apparent on the scan has an asymmetrical form allowing the front, rear, upper, and lower, and left/right defined surfaces that may be unambiguously determined from the analysis of the scan, thereby to allow the determination not only of the location of the fiducial reference, but also of its orientation.

In addition, the computer software may create a coordinate system for organizing objects in the scan, such as teeth, jaw bone, skin and gum tissue, other surgical instruments, etc. The coordinate system relates the images on the scan to the space around the fiducial and locates the instruments bearing markers both by orientation and position. The model generated by the monitoring system may then be used to check boundary conditions, and in conjunction with the tracker display the arrangement in real time on a suitable display, for example display 224 of FIG. 2.

In one embodiment, the computer system has a predetermined knowledge of the physical configuration of fiducial key 10 and examines slices/sections of the scan to locate fiducial key 10. Locating of fiducial key 10 may be on the basis of its distinct shape, or on the basis of distinctive identifying and orienting markings upon the fiducial key or on attachments to the fiducial key 10 as tracking marker 12. Fiducial key 10 may be rendered distinctly visible in the scans through higher imaging contrast by the employ of radio-opaque materials or high-density materials in the construction of the fiducial key 10. In other embodiments the material of the distinctive identifying and orienting markings may be created using suitable high density or radio-opaque inks or materials.

Once fiducial key 10 is identified, the location and orientation of the fiducial key 10 is determined from the scan segments, and a point within fiducial key 10 is assigned as the center of the coordinate system. The point so chosen may be chosen arbitrarily, or the choice may be based on some useful criterion. A model is then derived in the form of a transformation matrix to relate the fiducial system, being fiducial key 10 in one particular embodiment, to the coordinate system of the surgical site. The resulting virtual construct may be used by surgical procedure planning software for virtual modeling of the contemplated procedure, and may alternatively be used by instrumentation software for the configuration of the instrument, for providing imaging assistance for surgical software, and/or for plotting trajectories for the conduct of the surgical procedure.

In some embodiments, the monitoring hardware includes a tracking attachment to the fiducial reference. In the embodiment pertaining to dental surgery the tracking attachment to fiducial key 10 is tracking marker 12, which is attached to fiducial key 10 via tracking pole 11. Tracking marker 12 may have a particular identifying pattern. The trackable attachment, for example tracking marker 12, and even associated tracking pole 11 may have known configurations so that observational data from tracking pole 11 and/or tracking marker 12 may be precisely mapped to the coordinate system, and thus progress of the surgical procedure may be monitored and recorded. For example, as particularly shown in FIG. 3J, fiducial key 10 may have hole 15 in a predetermined location specially adapted for engagement with insert 17 of tracking pole 11. In such an arrangement, for example, tracking poles 11 may be attached with a low force push into hole 15 of fiducial key 10, and an audible haptic notification may thus be given upon successful completion of the attachment.

It is further possible to reorient the tracking pole during a surgical procedure. Such reorientation may be in order to change the location of the procedure, for example where a dental surgery deals with teeth on the opposite side of the mouth, where a surgeon switches hands, and/or where a second surgeon performs a portion of the procedure. For example, the movement of the tracking pole may trigger a re-registration of the tracking pole with relation to the coordinate system, so that the locations may be accordingly adjusted. Such a re-registration may be automatically initiated when, for example in the case of the dental surgery embodiment, tracking pole 11 with its attached tracking marker 12 are removed from hole 15 of fiducial key 10 and another tracking marker with its associated tracking pole is connected to an alternative hole on fiducial key 10. Additionally, boundary conditions may be implemented in the software so that the user is notified when observational data approaches and/or enters the boundary areas.

In a further embodiment of the system utilizing the invention, a surgical instrument or implement, herein termed a “hand piece” (see FIGS. 5 and 6), may also have a particular configuration that may be located and tracked in the coordinate system and may have suitable tracking markers as described herein. A boundary condition may be set up to indicate a potential collision with virtual material, so that when the hand piece is sensed to approach the boundary condition an indication may appear on a screen, or an alarm sound. Further, target boundary conditions may be set up to indicate the desired surgical area, so that when the trajectory of the hand piece is trending outside the target area an indication may appear on screen or an alarm sound indicating that the hand piece is deviating from its desired path.

An alternative embodiment of some hardware components are shown in FIGS. 3G-I. Fiducial key 10′ has connection elements with suitable connecting portions to allow a tracking pole 11′ to position a tracking marker 12′ relative to the surgical site. Conceptually, fiducial key 10′ serves as an anchor for pole 11′ and tracking marker 12′ in much the same way as the earlier embodiment, although it has a distinct shape. The software of the monitoring system is pre-programmed with the configuration of each particularly identified fiducial key, tracking pole, and tracking marker, so that the location calculations are only changed according to the changed configuration parameters.

The materials of the hardware components may vary according to regulatory requirements and practical considerations. Generally, the key or fiducial component is made of generally radio opaque material such that it does not produce noise for the scan, yet creates recognizable contrast on the scanned image so that any identifying pattern associated with it may be recognized. In addition, because it is generally located on the patient, the material should be lightweight and suitable for connection to an apparatus on the patient. For example, in the dental surgery example, the materials of the fiducial key must be suitable for connection to a plastic splint and suitable for connection to a tracking pole. In the surgical example the materials of the fiducial key may be suitable for attachment to the skin or other particular tissue of a patient.

The tracking markers are clearly identified by employing, for example without limitation, high contrast pattern engraving. The materials of the tracking markers are chosen to be capable of resisting damage in autoclave processes and are compatible with rigid, repeatable, and quick connection to a connector structure. The tracking markers and associated tracking poles have the ability to be accommodated at different locations for different surgery locations, and, like the fiducial keys, they should also be relatively lightweight as they will often be resting on or against the patient. The tracking poles must similarly be compatible with autoclave processes and have connectors of a form shared among tracking poles.

The tracker employed in tracking the fiducial keys, tracking poles and tracking markers should be capable of tracking with suitable accuracy objects of a size of the order of 1.5 square centimeters. The tracker may be, by way of example without limitation, a stereo camera or stereo camera pair. While the tracker is generally connected by wire to a computing device to read the sensory input, it may optionally have wireless connectivity to transmit the sensory data to a computing device.

In embodiments that additionally employ a trackable piece of instrumentation, such as a hand piece, tracking markers attached to such a trackable piece of instrumentation may also be light-weight; capable of operating in a 3 object array with 90 degrees relationship; optionally having a high contrast pattern engraving and a rigid, quick mounting mechanism to a standard hand piece.

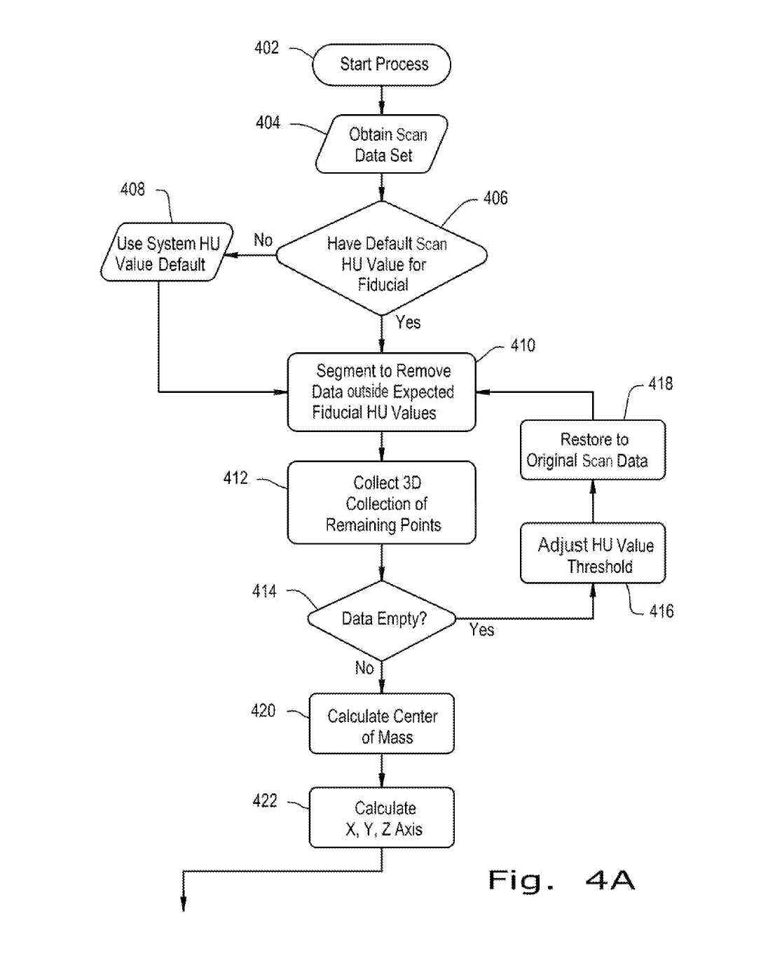

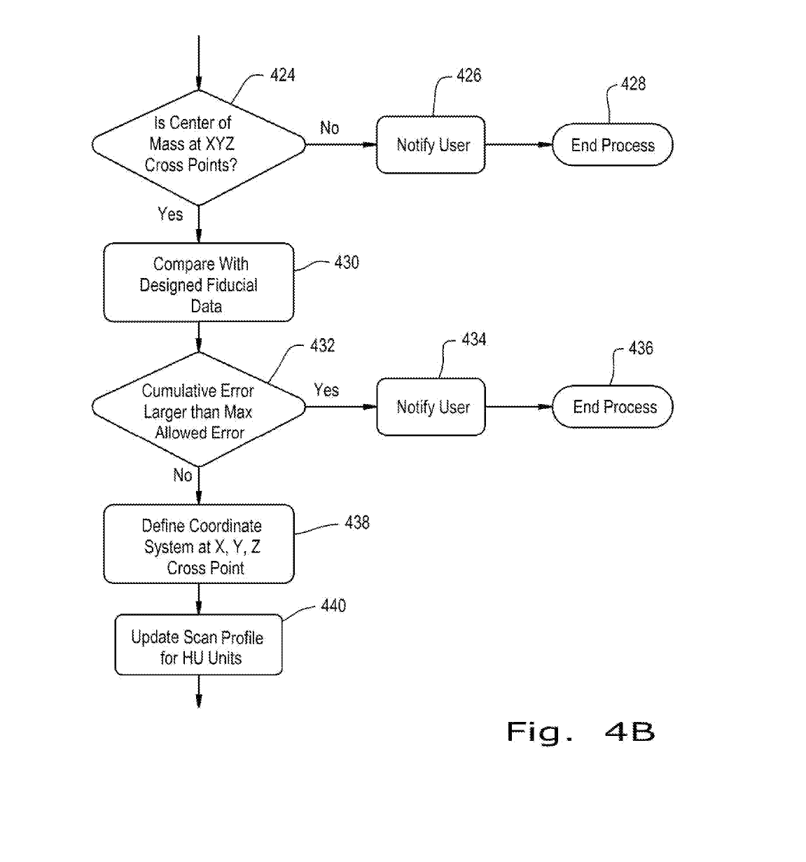

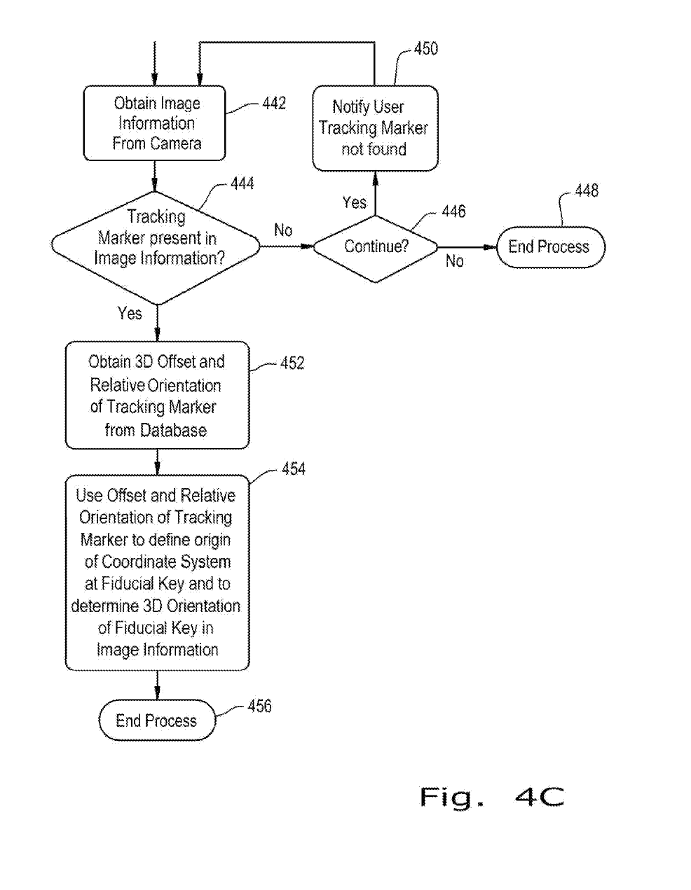

In another aspect there is presented an automatic registration method for tracking surgical activity, as illustrated in FIGS. 4A-C. FIG. 4A and FIG. 4B together present, without limitation, a flowchart of one method for determining the three-dimensional location and orientation of the fiducial reference from scan data. FIG. 4C presents a a flow chart of a method for confirming the presence of a suitable tracking marker in image information obtained by the tracker and determining the three-dimensional location and orientation of the fiducial reference based on the image information.

Once the process starts [402], as described in FIGS. 4A and 4B, the system obtains a scan data set [404] from, for example, a CT scanner and checks for a default CT scan Hounsfield unit (HU) value [at 406] for the fiducial which may or may not have been provided with the scan based on a knowledge of the fiducial and the particular scanner model, and if such a threshold value is not present, then a generalized predetermined default value is employed [408]. Next the data is processed by removing scan segments with Hounsfield data values outside expected values associated with the fiducial key values [at 410], following the collection of the remaining points [at 412]. If the data is empty [at 414], the CT value threshold is adjusted [at 416], the original value restored [at 418], and the segmenting processing scan segments continues [at 410]. Otherwise, with the existing data a center of mass is calculated [at 420], along with calculating the X, Y, and Z axes [at 422]. If the center of mass is not at the cross point of the XYZ axes [at 424], then the user is notified [at 426] and the process stopped [at 428]. If the center of mass is at the XYZ cross point then the data points are compared withe designed fiducial data [430]. If the cumulative error is larger than the maximum allowed error [432] then the user is notified [at 434] and the process ends [at 436]. If not, then the coordinate system is defined at the XYZ cross point [at 438], and the scan profile is updated for the HU units [at 440].

Turning now to FIG. 4C, an image is obtained from the tracker, being a suitable camera or other sensor [442]. The image information is analyzed to determine whether a tracking marker is present in the image information [444]. If not, then the user is queried [446] as to whether the process should continue or not. If not, then the process is ended [448]. If the process is to continue, then the user can be notified that no tracking marker has been found in the image information [450], and the process returns to obtaining image information [442]. If a tracking marker has been found based on the image information, or one has been attached by the user upon the above notification [450], the offset and relative orientation of the tracking marker to the fiducial reference is obtained from a suitable database [452]. The term “database” is used in this specification to describe any source, amount or arrangement of such information, whether organized into a formal multi-element or multi-dimensional database or not. A single data set comprising offset value and relative orientation may suffice in a simple implementation of this embodiment of the invention and may be provided, for example, by the user or may be within a memory unit of the controller or in a separate database or memory.

The offset and relative orientation of the tracking marker is used to define the origin of a coordinate system at the fiducial reference and to determine the three-dimensional orientation of the fiducial reference based on the image information [454] and the registration process ends [458]. In order to monitor the location and orientation of the fiducial reference in real time, the process may be looped back from step [454] to obtain new image information from the camera [442]. A suitable query point may be included to allow the user to terminate the process. Detailed methods for determining orientations and locations of predetermined shapes or marked tracking markers from image data are known to practitioners of the art and will not be dwelt upon here. The coordinate system so derived is then used for tracking the motion of any items bearing tracking markers in the proximity of the surgical site. Other registration systems are also contemplated, for example using current other sensory data rather than the predetermined offset, or having a fiducial with a transmission capacity.

One example of an embodiment of the invention is shown in FIG. 5. In addition to fiducial key 502 mounted at a predetermined tooth and having a rigidly mounted tracking marker 504, an additional instrument or implement 506, for example a hand piece which may be a dental drill, may be observed by a camera 508 serving as tracker of the monitoring system.

Another example of an embodiment of the invention is shown in FIG. 6. Surgery site 600, for example a human stomach or chest, may have fiducial key 602 fixed to a predetermined position to support tracking marker 604. Endoscope 606 may have further tracking markers, and biopsy needle 608 may also be present bearing a tracking marker at surgery site 600. Sensor 610, may be for example a camera, infrared sensing device, or RADAR.

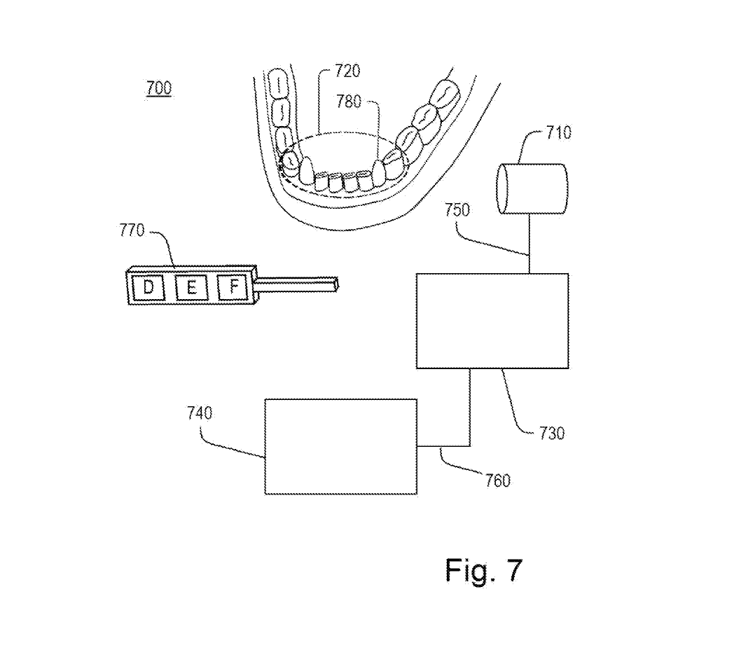

A further aspect of the invention involves embodiments of surgical monitoring systems and methods which allow for surgical planning while the patient is available for surgery, for example while the patient is being prepared for surgery, so that the system may model the surgical site. By way of comparison with the embodiments described in the foregoing, these embodiments identify the location and orientation of anatomical features within and items proximate a surgical site by employing as natural fiducial reference at least one identifiably distinctive anatomical feature. FIG. 7 presents a dental surgery embodiment wherein surgical monitoring system shown generally at 700 comprises tracker 710 for obtaining image information of surgical site 720, controller 730 for processing image information, and display 740 for displaying information from controller 730. Controller 730 may comprise, for example, processor 214 and memory 217 of computer 210 of FIG. 2. Display 740 may comprise, for example, display 224 of FIG. 2. Tracker 710 may be configured for supplying image information obtained from surgery site 720 to controller 730 via first interface connection 750. First interface connection 750 may be wired or wireless. Controller 730 may be configured for supplying display information to display 740 via second interface connection 760. Second interface connection 760 may be wired or wireless. Controller 730 and display 740 are shown schematically in FIG. 7.

Surgical monitoring system 700 may optionally comprise one or more independent instruments or items 770 bearing tracking markers of the type described in the foregoing embodiments. This allows independent items to be tracked by tracker 710. An example, without limitation, of instrument 770 for use in the dental surgery embodiment involves a dentist's drill.

In the dental surgery embodiment shown in FIG. 7, canine tooth 780, located within or proximate surgical site 720, is used, by way of example, as identifiably distinctive anatomical feature to serve as natural fiducial reference. A canine tooth is generally identifiably distinctive as compared with adjacent incisors or molars and lends itself to serving as natural fiducial reference. The term “natural fiducial reference” is used in the present specification to describe an identifiably distinctive anatomical feature including, without limitation, a canine tooth or other reliably distinctive feature.

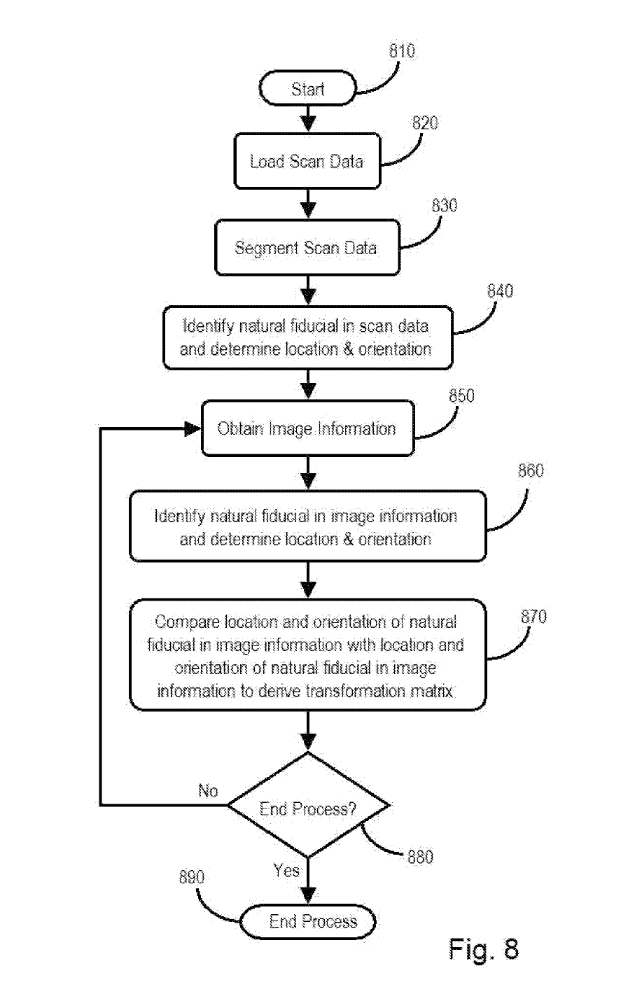

A further aspect of the present invention involves embodiments of an automatic registration method for tracking surgical activity, as illustrated in FIG. 8. Using the dental surgery example, surgical site 720 is scanned to obtain scan data. Alternatively, existing scan data may be employed. The scan data is typically, though not exclusively, three-dimensional in nature. It is therefore not typically available as a single two-dimensional image allowing a human viewer, for example a surgeon, to accurately determine the three-dimensional location and orientation of any natural fiducial reference. For human viewing, the sophisticated equipment used for obtaining the scans typically makes it possible to page through various two-dimensional “slices”. In yet more advanced systems a simulated three-dimensional presentation is done in two dimensions, typically allowing the simulated rotation of the scanned area on a computer display. These facilities, while impressive and helpful in diagnostic activities, do not enable human viewers to accurately determine three-dimensional locations and orientations of anatomical features using merely their eyes. More sophisticated computer-based interpretation of the scan data is required for such determination of three-dimensional locations and orientations of anatomical features to a suitable degree of accuracy.

The method of the present embodiment starts [at 810] with loading [at 820] of scan data into controller 730. The scan data is then segmented [at 830]. The identifiably distinctive configuration of canine tooth 780 allows computer software stored in memory and executed in controller 730 to identify canine tooth 780 in the scan data and to determine [at 840] its three-dimensional location and orientation within surgical site 720.

With the natural fiducial reference canine tooth 780 located and oriented within the scan data, tracker 710 is employed to obtain image information [at 850] of surgical site 720. The computer software stored in memory and executed in controller 730 then processes the image information to identify canine tooth 780 within the image information and determines [at 860] the location and orientation of canine tooth 780 within surgical site 720.

Many techniques are known in the field of computer vision for finding reference points in an image. By way of example, these include, but are not limited to, the Harris Corner Detector and the Scale Invariant Feature Transform (SIFT). These are commonly known as ‘good features to track’ and select points in an image that may be reliably recognized by a computer algorithm. These algorithms are commonly used to compare different images and determine the position of the same or similar object. These are then commonly compared with another image of the same scene to determine the motion of the object, or camera between the two views. Examples of specific algorithms for implementing techniques for finding such reference points may be found in the papers: C. Harris and M. Stephens (1988), “A combined corner and edge detector,” Proceedings of the 4th Alvey Vision Conference. pp. 147-151; and Lowe, David G. (1999). “Object recognition from local scale-invariant features”. Proceedings of the International Conference on Computer Vision. 2. pp. 1150-1157, the disclosures of which are explicitly incorporated by reference herein.

The present invention extends this concept to compare a two-dimensional view of surgical site 720 as obtained from tracker 710 with the existing three-dimensional scan data to identify canine tooth 780 within the image information and determines [at 860] the location and orientation of canine tooth 780 within surgical site 720. In one exemplary embodiment, a number of distinct points are identified from the existing three-dimensional scan data. The points may be chosen on the basis of geometrical properties of the edges, separation from other material types identified in the scan data, the distribution of positions through the scan data volume. A number of two-dimensional projections of these points may be made onto a number of arbitrary planes and the two-dimensional maps may be compared to the image information obtained from tracker 710. From the relative orientations of corresponding points in the image information and the generated maps it is possible, with sufficient number of point correspondences, to determine a relative position and orientation of tracker 710 compared with the chosen plane.

In another embodiment, image information in the form of a two-dimensional image may be obtained from tracker 710 and the relative locations of potential good features to track may be determined. Distinct points are then identified from the existing three-dimensional scan data and possible projections to match the two-dimensional image may be calculated. When a match is made with corresponding points the relative orientation and position of tracker 710 and the three-dimensional scan data may then be determined.

Since the sets of data representing the three-dimensional location and orientation of canine tooth 780 within the scan data and within the image information are available to the computer software, the two sets of data are compared [at 870] to derive a three-dimensional spatial transformation matrix between the location and orientation of canine tooth 780 in the scan data, and the same tooth in the mage information. The three-dimensional transformation matrix therefore in general relates a three-dimensional coordinate in the image information to a corresponding three-dimensional coordinate in the scan data. By looping the process back [at 880] to obtain refreshed image information, controller 730 may monitor the three-dimensional location and orientation of canine tooth 780 in real time.

The computer software may create a coordinate system for organizing objects in the scan, such as other teeth, jaw bone, skin and gum tissue, and other surgical instruments, including one or more instruments or items 770 bearing suitable tracking markers. The coordinate system relates the scan data to surgical site 720 around canine tooth 780 and optionally locates any instruments bearing markers as described in the foregoing embodiments, both by three-dimensional orientation and three-dimensional position. A model of surgical site 720 may be generated by monitoring system 700 and may then be used to check boundary conditions. In conjunction with tracker 710, controller 730 may display the arrangement in real time on display 740.

The model created by the software may be used to track contemplated surgical procedures and alert the physician to possible boundary violations that would indicate an inappropriate location in a surgical procedure, for example, when the at least one instrument 770 is determined to be proximate an inappropriate location within surgical site 720.

In further embodiments, at least one of items or instruments 770 near surgical site 720 may optionally be tracked by tracker 710 and controller 730 of surgical monitoring system 700 based on its three-dimensional shape rather than due to the presence of a marker. In these embodiments, controller 730 may be pre-programmed with data describing the three-dimensional shape of item or instruments 770, which refer to for these purposes as an “object” (770).

In yet further embodiments, at least one of items or instruments near surgical site 720 may optionally have a tracker attached to function as tracker for monitoring system 700 and to thereby sense the orientation and the position of the tracking marker and of any other additional tracking markers relative to the scan data of the surgical area. By way of example, the tracker attached to instrument 770 may involve a miniature digital camera and instrument 770 may involve a dentist's drill. Any other markers to be tracked by the tracker attached to the instrument must be within the field of view of the tracker.

In should be pointed out that the application of the apparatus and method is not confined to actual living patients, human or animal, and may be applied in demonstration situations to various models, in which case the natural fiducial reference employed is an identifiably distinctive feature of the model employed for the demonstration, whether or not such a feature is “natuiral.”

The approach described in this embodiment requires only one visit to a professional, that being the visit to obtain the scan of surgical area 720. Such an approach obviates the need for specialized fiducial references and the need for further visits to professionals for the measuring, making or fitting of any splints or fiducial structures.

In yet a further embodiment of the surgical monitoring system, a user, who may be a surgeon, may attach a fixed tracking marker proximate the surgical site immediately before surgery. The tracking marker may be rigidly attached to the surgical site. The arrangement can be exactly the same as in FIGS. 3 A-J, FIG. 5 and FIG. 6, with the specific difference that item 10, 10′, 502, 602 of those figures is not a fiducial reference in this embodiment, but merely a rigid mount for attaching to the surgical site. The surgical monitoring system of this embodiment does not employ any fiducial reference that is to be located in both scan data and image information. However, as in the embodiments described relating to FIGS. 3A-J, FIG. 5 and FIG. 6, the rigid mount 10, 10′, 502, 602 may contain visible marker features that allow its position and orientation to be determined by the tracking system from image information gathered by the tracker. The surgical monitoring system may also employ tracking pole 11, 11′ to attach tracking marker 12, 12′, 504, 604 to rigid mount 10, 10′, 502, 602.

At various points during the surgical procedure the location and orientation of tracking marker 12, 12′, 504, 604 may be determined, along with recognized natural features within the surgical site. Since the location and orientation of tracking marker 12, 12′, 504, 604 relative to rigid mount 10, 10′, 502, 602 is fixed, the knowledge of the location and orientation of tracking marker 12, 12′, 504, 604 may then be employed to determine the three dimensional location and orientation of rigid mount 10, 10′, 502, 602 relative to the surgical scan without further reference to the natural fiducial features. In this way it is possible to continually orient the tracking system to the surgical data if the natural fiducial features are no longer detectable or if they cannot be detected with sufficient precision.

In this way the natural fiducial features only have to be detected in a single instance and their position and orientation relative to the surgical scan need only be determined once. This embodiment allows for the surgical monitoring system to track the surgical procedure in real time even when detection of fiducial features, natural or otherwise are not imageable by the tracker. It also allows for continuity when such fiducial features are only sporadically imageable by the tracker.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A surgical monitoring system for observation of a surgical site, the system comprising a tracker having sensory equipment for observing proximate a surgical site; and a computing device in communication with the tracker and having a processor, said processor having access to scan data of the surgical site and to software that when executed by said processor enables said processor to:
recognize a natural fiducial reference in image information obtained by the tracker from the surgical site; and
calculate a model of the surgical site based on the image information and on the previous scan data.

2. The surgical monitoring system of claim 1 wherein the software when executed by said processor also enables said processor to recognize a natural fiducial in the scan data of the surgical site.

3. The surgical monitoring system of claim 1 further comprising a tracking marker attached proximate the surgical site wherein the software when executed by said processor enables said processor to:
determine a three-dimensional location and orientation of the tracking marker in the image information; and
determine from the three-dimensional location and orientation of the tracking marker a three-dimensional location and orientation of the natural fiducial reference in the image information.

4. A surgical monitoring system comprising a tracker for obtaining image information of a surgical site and a controller configured with:
means for obtaining image information of the surgical site from the tracker;
means for identifying a natural fiducial reference in the image information and in previously obtained scan data of the surgical site;
means for determining the three-dimensional location and orientation of the natural fiducial reference in the image information and in the scan data; and
means for comparing the three-dimensional location and orientation of the natural fiducial reference in the image information with the three-dimensional location and orientation of the natural fiducial reference in the scan data to spatially relate in three-dimensions the image information to the scan data, to derive a three-dimensional spatial transformation matrix that relates a three-dimensional coordinate in the image information to a corresponding three-dimensional coordinate in the scan data.

5. The surgical monitoring system of claim 4 further comprising at least one object, wherein the controller is further configured with:
means for identifying within the image information the at least one object based on pre-programmed data describing the three-dimensional shape of the at least one object; and
means for determining the three-dimensional location and orientation of the at least one object based on the pre-programmed data.

6. The surgical monitoring system of claim 4 further comprising at least one object bearing a tracking marker, wherein the controller is further configured with means for identifying within the image information the tracking marker and determining the three-dimensional location and orientation of the at least one object from information about the tracking marker.

7. The surgical monitoring system of claim 6 wherein the tracker is attached to one of the at least one objects.

8. The surgical monitoring system of claim 4 wherein the controller is configured with means for deriving the three-dimensional spatial transformation matrix in real time.

9. The surgical monitoring system of claim 4 further comprising a tracking marker attachable to the surgical site, wherein the controller is further configured with:
means for determining the three-dimensional location and orientation of the tracking marker in the image information; and
means for determining from the three-dimensional location and orientation of the tracking marker the three-dimensional location and orientation of the natural fiducial reference in the image information.

10. The surgical monitoring system of claim 9 wherein the tracking marker is attached to the surgical site by at least one of a tracking pole and a mount.

11. A method for relating in real time the three-dimensional location and orientation of a surgical site to the location and orientation of the surgical site in a scan of the surgical site, the method comprising the steps of performing a scan of the surgical site to obtain scan data; determining the three-dimensional location and orientation of a natural fiducial reference from the scan data; obtaining real time image information of the surgical site; determining in real time the three-dimensional location and orientation of the natural fiducial reference from the image information; deriving a spatial transformation matrix for expressing in real time the three-dimensional location and orientation of the natural fiducial reference as determined from the image information in terms of the three-dimensional location and orientation of the natural fiducial reference as determined from the scan data.

12. A method for relating the three-dimensional location and orientation of a surgical site to the location and orientation of the surgical site in a scan of the surgical site, the method comprising the steps of:
obtaining image information of the surgical site from the tracker;
identifying a natural fiducial reference in the image information and in previously obtained scan data of the surgical site;
determining the three-dimensional location and orientation of the natural fiducial reference in the image information and in the scan data;
comparing the three-dimensional location and orientation of the natural fiducial reference in the image information with the three-dimensional location and orientation of the natural fiducial reference in the scan data to spatially relate in three-dimensions the image information to the scan data to derive a three-dimensional spatial transformation matrix that relates a three-dimensional coordinate in the image information to a corresponding three-dimensional coordinate in the scan data.

13. The method of claim 12 further comprising the step of creating a model of the surgical site.

14. The method of claim 13 further comprising the steps of:
identifying within the image information a tracking marker attached to at least one instrument proximate the surgical site; and
determining the three-dimensional location and orientation of the at least one object from information about the tracking marker.

15. The method of claim 14 further comprising the step of issuing an alert when the at least one instrument is determined to be proximate an inappropriate location within the surgical site.

16. The method of claim 12 wherein the comparing step includes deriving the three-dimensional spatial transformation matrix in real time by obtaining refreshed image information in real time.

17. The method of claim 12 wherein the tracker is attached to one of the at least one objects.

18. A method for tracking at least one object proximate a surgical site, comprising the steps of:
performing a scan of the surgical site to obtain scan data;
determining the three-dimensional location and orientation of a natural fiducial reference from the scan data;
obtaining real time image information of the surgical site;
determining in real time the three-dimensional location and orientation of the natural fiducial reference from the image information;
deriving a spatial transformation matrix for expressing in real time the three-dimensional location and orientation of the natural fiducial reference as determined from the image information in terms of the three-dimensional location and orientation of the natural fiducial reference as determined from the scan data;
identifying within the image information the at least one object based on pre-programmed data describing the three-dimensional shape of the at least one object; and
determining the three-dimensional location and orientation of the at least one object relative to the three-dimensional location and orientation of the surgical site based on the pre-programmed data.

19. The method of claim 18 further comprising the steps of:
determining in the image information a three-dimensional location and an orientation of a tracking marker attached to the surgical site; and
determining from the three-dimensional location and orientation of the tracking marker the three-dimensional location and orientation of the natural fiducial reference in the image information.

20. The method of claim 19 wherein the tracking marker is attached to the surgical site via at least one of a tracking pole and a mount.
